(19) United States
(12) Patent Application Publication
(10) Pub. No.: US 2012/0158135 A1
(43) Pub. Date: Jun. 21, 2012

(54) COUPLING APPARATUS

(75) Inventors: Eric William Abel, Dundee (GB); Adam Ramage, Perthshire (GB)

(73) Assignee: SENTIENT MEDICAL LIMITED, Dundee (GB)

(21) Appl. No.: 13/380,755

(22) PCT Filed: Jun. 24, 2010
(86) PCT No.: PCT/GB2010/051046

§ 371 Date: Mar. 12, 2012

(30) Foreign Application Priority Data
Jun. 24, 2009 (GB) 0910906.7

(51) Int. Cl. A61F 2/18

(52) U.S. Cl. 623/10

(57) ABSTRACT

Coupling apparatus for coupling an implantable element to the round window membrane. The apparatus comprises engagement means in the form of a clip or a filler material for engaging the bone surface within the round window niche. This supports the apparatus in the region of the round window membrane.

The present invention relates to coupling apparatus, and more particularly to coupling apparatus for coupling a middle ear implant to the stapes footplate.

The term “middle ear” refers to the tympanic cavity, located between the external auditory canal and the cochlea.

In a healthy ear, vibrations of the tympanic membrane, or ear drum, which is located at the boundary between the auditory canal and the tympanic cavity, are communicated across the tympanic cavity to the cochlea by a series of three articulated bones known as the ossicular chain.

The ossicular chain comprises three individual ossicles. Namely, the malleus, the incus and the stapes. The malleus is connected between the tympanic membrane and the incus. The incus is in turn connected between the malleus and the stapes. The stapes comprises a footplate portion which is disposed against a membrane which covers an opening to the cochlea, known as the oval window.

Vibrations of the tympanic membrane are thus transmitted by the ossicles to the oval window membrane, to cause pressure variations within the fluid filled cochlea. These pressure variations are accommodated by the presence of a second membrane covered opening, known as the round window, such that the round window membrane vibrates in counter-phase with the oval window membrane.

The term “middle ear implant” refers generally to devices which can be implanted into the tympanic cavity of patients with sensorineural or conductive hearing loss, to improve their hearing.

Sensorineural hearing loss is attributable to defects in the inner ear which reduce its ability to convert vibrational stimulus into neural activity and/or to defects in the parts of the nervous system associated with hearing.

Conductive hearing loss is attributable to defects in the conductive elements of the middle ear, ie the ossicular chain, which prevent the effective conduction of vibrational energy across the middle ear cavity.

In both cases, the patient=s hearing can be improved by amplifying the vibrational stimulus applied to the inner ear by introducing a hearing actuator for actively vibrating one or more elements within the middle ear in response to an external signal from a microphone or other sensor.

In the case of conductive hearing loss, the patient=s hearing can also be improved by replacing or bypassing all or part of the ossicular chain with a prosthesis, which acts as a conductive bridge.

Such devices are collectively referred to as middle ear implants. Implants which conduct vibrational energy across the middle ear, without themselves generating vibrational energy, are referred to as passive implants. Implants which themselves generate vibrational energy are referred to as active implants. Some middle ear implants may comprise both passive and active elements.

A multitude of different middle ear implants, which extend between a variety of different elements within the middle ear, have been developed.

However, it is particularly desirable for an implant to be coupled to the footplate of the stapes, which lies against the oval window membrane, and thus conducts vibrations directly to the fluid filled cochlea.

WO 2008/139225 describes a middle ear implant of the active type, which extends from the incus long process to the footplate of the stapes, such that vibrations generated by the implant are conveyed to the stapes footplate, and thus to the cochlea.

The implant is coupled at one end to the incus long process by means of a spring clip. However, coupling the other end of the implant to the stapes footplate is not straightforward, due to the generally flat form of the footplate. In WO 2008/139225, the implant comprises a rod, which extends from the end of the transducer and presses against the footplate, where it is retained in place by friction. Whilst this is generally effective, there remains a risk that the rod will slip to a position on the footplate where vibrations are transmitted less effectively, or that it will lose contact with the footplate altogether.

The risk of the contacting portion slipping can be avoided by securing the rod to the footplate by mechanical means such as screws or using bio-compatible adhesive. However, this requires intricate and time consuming surgery, the effects of which are not easily reversed if and when the implant needs to be removed.

It is an object of the present invention to overcome the aforementioned problems.

According to one aspect of the present invention, there is provided an implantable device for implantation in the middle ear, the device comprising engagement means for engaging the footplate of the stapes, the engagement means comprising:—

a first coupling portion configured for location on the footplate of the stapes; and

a second coupling portion for coupling to the first coupling portion;

wherein one of the first and second coupling portions comprises a projection, and the other of said portions comprises a correspondingly formed opening for receiving the projection, to provide a pivotal coupling between said first and second coupling portions.

With this arrangement, the implantable device can be securely mounted to the stapes footplate through the location of the first coupling portion on the stapes footplate and through the coupling of the first coupling portion with the second coupling portion. Moreover, the pivotal coupling between the first and second coupling portions means that the angle of the second coupling portion with respect to the first coupling portion can be adjusted to facilitate implantation of the device.

In preferred embodiments, the projection is formed on the second coupling portion and the corresponding opening is formed on the first coupling portion. That is to say, the opening is preferably formed on the part which contacts the stapes footplate. However, the opening may alternatively be formed on the second coupling portion, with the projection formed on the first coupling portion.

The projection or the opening is preferably formed centrally on the first coupling portion.

Thus, when the first coupling portion is centrally mounted to the stapes footplate, the second coupling portion can be located substantially equidistant from the stapes arches. Vibrations may be more effectively conducted to the stapes footplate and the oval window with the second coupling portion located substantially equidistant from the stapes arches.

The projection is preferably rounded. In particular, the projection preferably has a substantially constant radius of curvature. More preferably, the projection has a substantially hemispherical form.

The portion on which the projection is formed may comprise an annular groove adjacent the projection. This increases the range of angular adjustment of the second coupling portion in relation to the first coupling portion.

The opening preferably has a substantially constant radius of curvature. More preferably, the opening has a substantially hemispherical form. In both cases, the radius of curvature of the projection is preferably fractionally smaller than the radius of curvature of the opening. This allows the surface of the projection to slide relative to the surface of the recess, whilst the projection is securely retained by the recess.

Alternatively, the opening may have a substantially cylindrical form. A cylindrical opening can cooperate with an appropriately formed projection such as one having a rounded or hemi-spherical form, to allow pivotal movement of the second coupling portion with respect to the first coupling portion.

The second coupling portion may be an elongate portion such as a rod. In this case the projection or the opening is preferably formed at an end of the elongate portion. In the case where the projection is formed on the elongate portion, this may simply be a rounded end thereof.

The first coupling portion may be configured to engage the surface of the stapes footplate through friction. In this case, the first coupling portion preferably has a surface configured for contacting a relatively large proportion of the exposed surface of the stapes footplate. For example, at least 30% of the exposed surface of the footplate, more preferably at least 50% thereof. In such cases, surface tension due to moisture on the footplate may assist in retaining the first coupling portion on the footplate.

Where the plate is made of titanium or other bioactive material which encourages bone growth, bone may, over time, grow to the plate to hold it in position.

Alternatively, or in addition, the implantable device may comprise an attachment means for attaching the first coupling portion to the stapes footplate. The first coupling portion may be mounted or mountable to the attachment means, or may be integrally formed therewith.

The attachment means preferably comprises first and second engagement sections connected by a connecting portion, wherein each of the engagement sections comprises an engagement surface which is configured to engage a respective one of the stapes arches.

The attachment means can thus be attached to the stapes by engaging the engagement sections with the stapes arches. When attached to the stapes in this way, the connecting portion extends over the stapes footplate, and thus allows the first coupling portion to be coupled to the footplate.

The engagement surface of the first engagement section preferably faces in an opposite direction to the engagement surface of the second engagement section. Thus, the engagement sections will press against the stapes arches in opposite directions to grip the arches securely.

The attachment means may be configured such that the engagement surfaces face towards one another. In this case, the attachment means will press against the outward facing surfaces of the stapes arches. Alternatively, the attachment means may be configured such that the concave surfaces face away from one another. In this case, the attachment means will press against the inward facing surfaces of the stapes arches.

The engagement surfaces of the engagement sections are preferably spaced apart by a distance substantially equal to the distance between the stapes arches where they meet the stapes footplate. Thus, the attachment means is configured to be mounted to the stapes in a region adjacent the stapes footplate.

Preferably, the engagement sections each comprise a curved section, and the respective engagement surfaces are preferably concave surfaces.

The attachment means is preferably at least partially resilient, to be resiliently deformable between a first configuration for insertion through the stapes arches or passing around the stapes arches, and a second configuration for engaging the stapes arches.

In particular, the connecting portion of the attachment means preferably comprises at least one resilient section. This allows the attachment means to be resiliently deformed such that the engagement sections can be passed around or inserted between the stapes arches before being released to grip the arches.

The connecting portion may comprise two resilient sections, located one on each side of a central region of the connecting portion. This allows the central region of the connecting portion to be mounted to the first coupling component, without affecting the resiliency of the attachment means. The first coupling portion may be mounted or mountable to the connecting portion, or may be integrally formed therewith.

The attachment means preferably has super-elastic properties. In this respect, attachment means is preferably at least partially formed of a super-elastic material. The material from which the attachment means is formed is preferably a nickel titanium alloy, such as Nitinol, or some other alloy or polymer or other material with super-elastic properties.

Moreover, the attachment means is preferably configured such that the deflection between its natural or original configuration, and a second configuration in which the engagement sections grip the stapes arches, is sufficiently large, that the engagement sections will operate super-elastically to grip the stapes arches. That is to say, the force exerted by the engagement sections on the stapes arches will be substantially constant over a wide range of deflections. This is desirable because it means that an attachment means of a given size can accommodate significant variation in stapes size between patients.

The attachment means is preferably a clip.

In a preferred embodiment, the first and second engagement sections extend in a first plane, and the connecting portion extends from the engagement sections in a second plane perpendicular to the first plane. A central portion of the connecting portion preferably coincides with the intersection of the first and second planes.

The connecting section preferably comprises at least one curved section, more preferably two curved sections connected together centrally of the attachment means. In a particularly preferred embodiment, the connecting portion of the clip may be substantially M-shaped. Such configurations enable the attachment means to grip the stapes arches super-elastically.

The connecting section preferably comprises a pair of outer legs, respectively connected to the engagement sections. These legs preferably form a cantilever like configuration. The length of each of these legs is preferably substantially greater than the cross-section dimension of the leg. More preferably, between 10 and 50 times greater.

The device is preferably configured such that the opening or projection of the first coupling portion is located substantially equidistant from the engagement surfaces of the attachment means.

Accordingly, the projection/opening, and thus the second coupling portion, can be located at a position substantially equidistant from the stapes arches. Vibrations may be more effectively conducted to stapes footplate and the oval window with the second coupling portion located substantially equidistant from the stapes arches.

The opening or projection (or its central point) is desirably offset from the central point between the engagement surfaces.

Accordingly, when the device is mounted to the stapes, the projection/opening will not be located immediately beneath the stapes arches.

Where the projection/opening is located beneath the stapes arches, the top of the stapes interferes with the path of the implant, meaning that the second coupling portion has to be angled away from the perpendicular to avoid the top of the stapes. This reduces the efficiency of transmission of vibration, because the component of force perpendicular to the second coupling portion is reduced.

With the opening/projection offset, the angle of contact between the first and second coupling portions can be substantially 90 degrees, to substantially maximise the component of force perpendicular to the second coupling portion, and improve the efficiency of transmission of vibration.

The device is preferably formed from a biocompatible material.

According to a second aspect of the present invention, there is provided attachment means for attaching an implantable device to the stapes footplate, the attachment means comprising first and second engagement sections connected by a connecting portion, wherein each of the engagement sections is configured to engage a respective one of the stapes arches.

The attachment means can thus be attached to the stapes by engaging the engagement sections with the stapes arches. When attached to the stapes in this way, the connecting portion extends over the stapes footplate, and thus allows an implantable device to be coupled to the footplate.

An engagement surface of the first engagement section preferably faces in an opposite direction to an engagement surface of the second engagement section. Thus, the engagement sections will press against the stapes arches in opposite directions to grip the arches securely.

The attachment means may be configured such that the engagement surfaces face towards one another. In this case, the attachment means will press against the outward facing surfaces of the stapes arches.

Although this is generally effective, there is a possibility that with some configurations, the attachment means may ride up the stapes arches, losing contact with the stapes footplate, and reducing its grip on the arches. This may be avoided by providing an attachment means which is configured such that the concave surfaces face away from one another. In this case, the attachment means will press against the inward facing surfaces of the stapes arches.

The engagement surfaces of the engagement sections are preferably spaced apart by a distance substantially equal to the distance between the stapes arches where they meet the stapes footplate. Thus, the attachment means may be configured to be mounted to the stapes in a region adjacent the stapes footplate.

Preferably, the engagement sections each comprise a curved section, and the respective engagement surfaces are preferably concave surfaces.

The attachment means is preferably at least partially resilient, to be resiliently deformable between a first configuration for insertion through the stapes arches or passing around the stapes arches, and a second configuration for engaging the stapes arches.

In particular, the connecting portion of the attachment means preferably comprises at least one resilient section. This allows the attachment means to be resiliently deformed such that the engagement sections can be passed around or inserted between the stapes arches before being released to grip the arches.

The connecting portion may comprise two resilient sections, located one on each side of a central region of the connecting portion. This allows the central region of the connecting portion to be mounted to a further component, without affecting the resiliency of the attachment means.

The attachment means preferably has super-elastic properties. In this respect, attachment means is preferably at least partially formed of a super-elastic material. The material from which the attachment means is formed is preferably a nickel titanium alloy, such as Nitinol, or some other alloy or polymer or other material with super-elastic properties.

Moreover, the attachment means is preferably configured such that the deflection between its natural or original configuration, and a second configuration in which the engagement sections grip the stapes arches, is sufficiently large, that the engagement sections will operate super-elastically to grip the stapes arches. That is to say, the force exerted by the engagement sections on the stapes arches will be substantially constant over a wide range of deflections. This is desirable because it means that an attachment means of a given size can accommodate significant variation in stapes size between patients.

The attachment means is preferably a clip.

In a preferred embodiment, the first and second engagement sections extend in a first plane, and the connecting portion extends from the engagement sections in a second plane perpendicular to the first plane. A central portion of the connecting portion preferably coincides with the intersection of the first and second planes.

The connecting section preferably comprises at least one curved section, more preferably two curved sections connected together centrally of the attachment means. In a particularly preferred embodiment, the connecting portion of the clip may be substantially M-shaped. Such configurations enable the attachment means to grip the stapes arches super-elastically.

The connecting section preferably comprises a pair of outer legs, respectively connected to the engagement sections. These legs preferably form a cantilever like configuration. The length of each of these legs is preferably substantially greater than the cross-section dimension of the leg. More preferably, between 10 and 50 times greater.

In a preferred embodiment, the attachment means may further comprise a footplate engaging portion for location on the footplate of the stapes.

Vibrations conducted to or generated by the footplate engaging portion are thus conducted directly to the stapes footplate.

The footplate engaging portion may be bonded or otherwise mounted to the connecting portion, or may be integrally formed therewith.

The footplate engaging portion may comprise one of an opening and a projection for receiving a correspondingly formed opening or projection of a coupling portion, to form a pivotal coupling with said coupling portion.

The attachment means is preferably configured such that said opening or projection is located substantially equidistant from the first and second engagement surfaces.

Accordingly, the projection/opening, and thus the coupling portion, can be located at a position substantially equidistant from the stapes arches. Vibrations may be more effectively conducted to the stapes footplate and the oval window with the coupling portion located substantially equidistant from the stapes arches.

The opening or projection (or its central point) is desirably offset from the central point between the engagement surfaces. Accordingly, when the attachment means is mounted to the stapes, the projection/opening will not be located immediately beneath the stapes arches.

The attachment means may further comprise a coupling portion for coupling with the footplate engaging portion, wherein the coupling portion comprises said correspondingly formed opening or projection for forming the pivotal connection with the footplate engaging portion.

In preferred embodiments, the projection is formed on the footplate engaging portion and the opening is formed on the coupling portion. However, the opening may alternatively be formed on the coupling portion, with the projection formed on the footplate engaging portion.

The projection is preferably rounded. In particular, the projection preferably has a substantially constant radius of curvature. More preferably, the projection has a substantially hemispherical form.

The portion on which the projection is formed may comprise an annular groove adjacent the projection. This increases the range of angular adjustment of the second coupling portion in relation to the first coupling portion.

The opening preferably has a substantially constant radius of curvature. More preferably, the opening has a substantially hemispherical form. In both cases, the radius of curvature of the projection is preferably fractionally larger than the radius of curvature of the opening. This allows the surface of the projection to slide relative to the surface of the recess, whilst the projection is securely retained by the recess.

Alternatively, the opening may have a substantially cylindrical form.

The coupling portion may comprise an elongate portion such as a rod. In this case the projection or the opening is preferably formed at an end of the elongate portion. In the case where the projection is formed on the elongate portion, this may simply be a rounded end thereof.

The attachment means is preferably formed from a bio-compatible material.

According to a further aspect of the present invention, there is provided a method of mounting an implantable device to the stapes footplate, the method comprising:—

locating a first coupling portion on the stapes footplate;

locating a second coupling portion on the first coupling portion, to be pivotally coupled therewith through engagement of a projection formed on one of said first and second coupling portions and a correspondingly formed opening formed in the other of said coupling portions; and

adjusting the angle of the second coupling portion to a desired position.

The method may further comprise the step of attaching the first coupling portion to the stapes footplate with an attachment means.

According to a further aspect of the present invention, there is provided a method of attaching an implantable device to the stapes footplate, the method comprising:—

providing an attachment means having first and second engagement sections, each configured to engage a respective one of the stapes arches; and

locating the first and second engagement sections around the respective stapes arch adjacent the stapes footplate.

In the case where the attachment means is resiliently deformable, the method may further comprise resiliently deforming the attachment means to pass around or through the stapes arches, and releasing the attachment means such that the engagement sections grip the arches.

The present invention will now be described with reference to the accompanying drawings in which:—

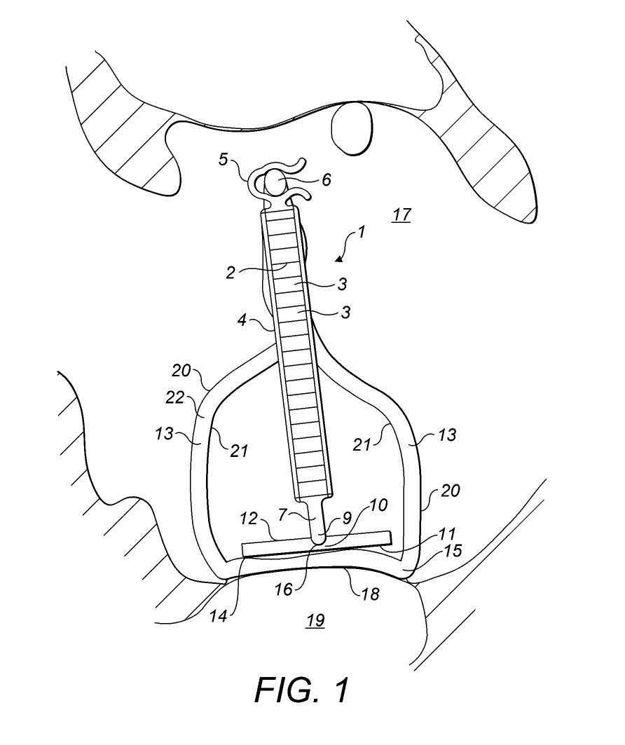

FIG. 1 shows a middle ear implant which embodies a first aspect of the present invention;

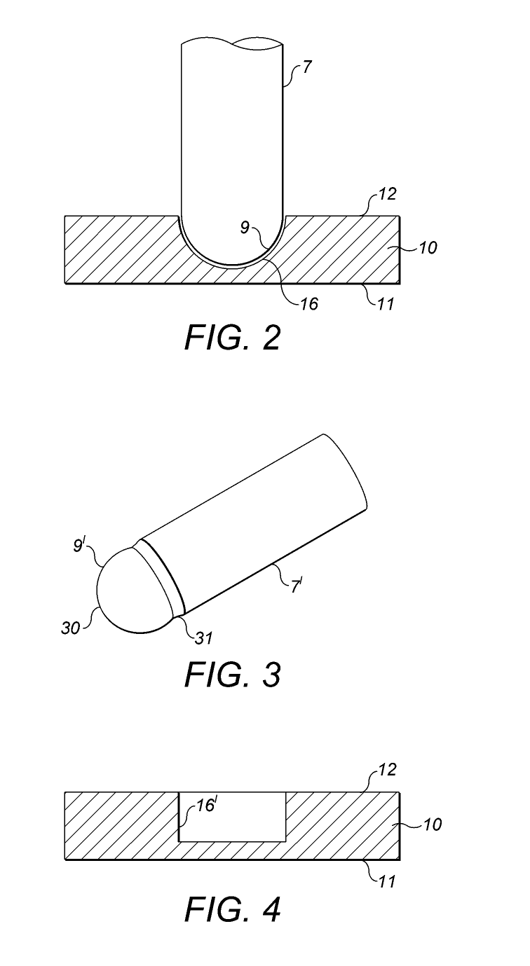

FIG. 2 is an enlarged cross-sectional view of the rod and plate of the implant shown in FIG. 1;

FIG. 3 shows an alternative form of the rod;

FIG. 4 shows an alternative form of the plate;

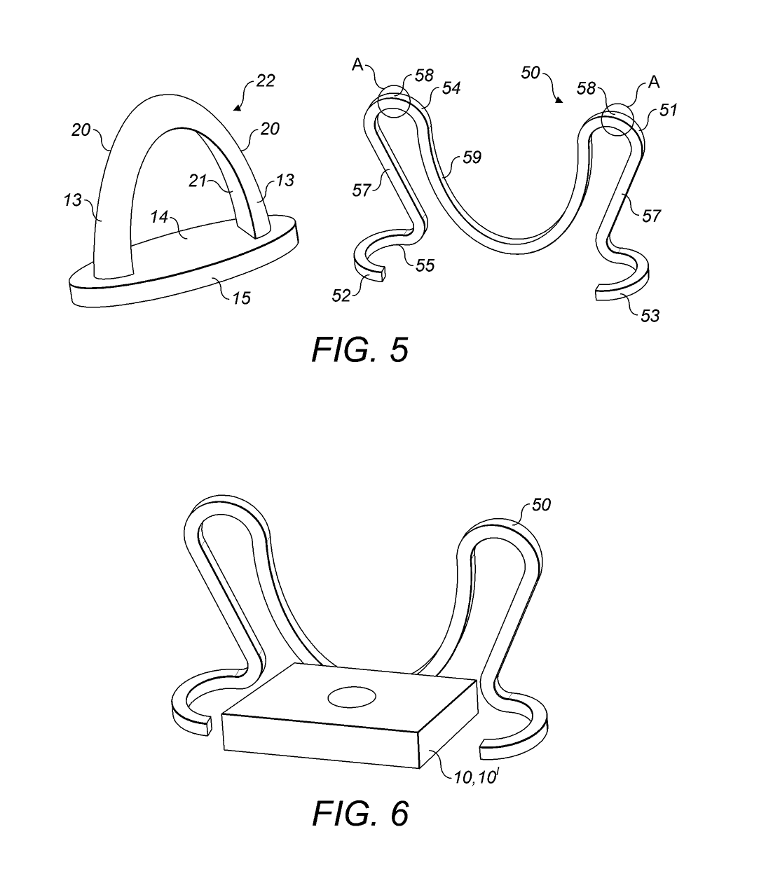

FIG. 5 shows the stapes alongside a clip which embodies a second aspect of the present invention, and which may form part of an implantable device which embodies the first aspect of the present invention;

FIG. 6 shows the clip of FIG. 1 mounted to the plate shown in FIGS. 1 and 2 or FIG. 4;

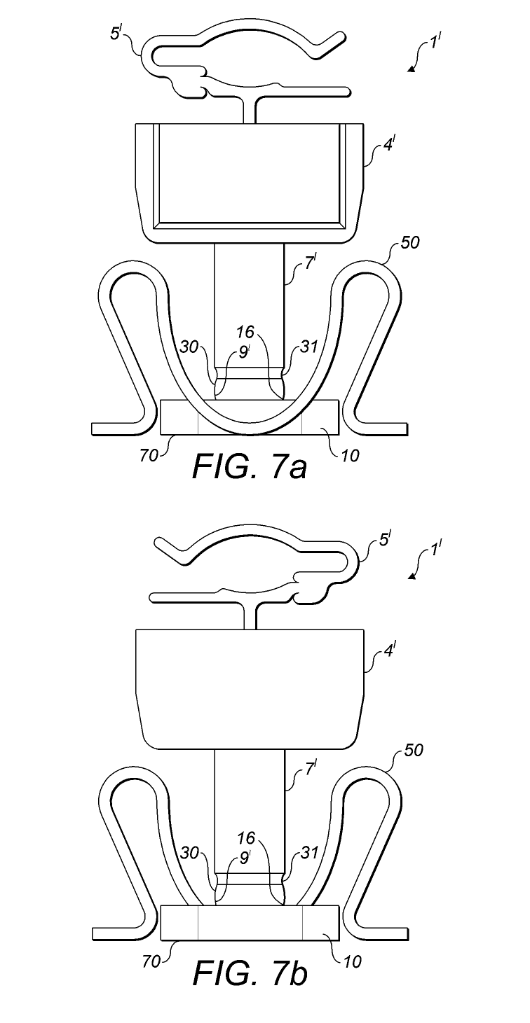

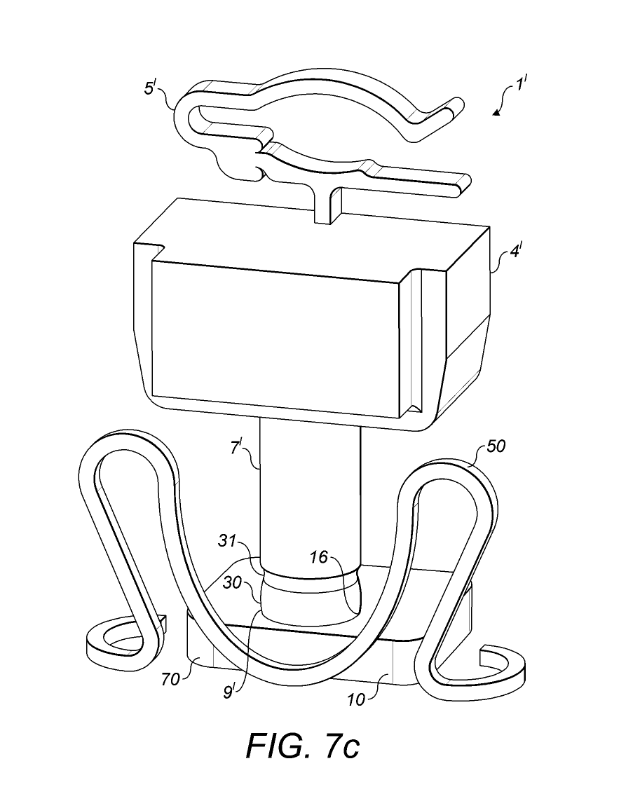

FIGS. 7a to 7c respectively show front, rear and perspective views of a second middle ear implant which embodies the present invention;

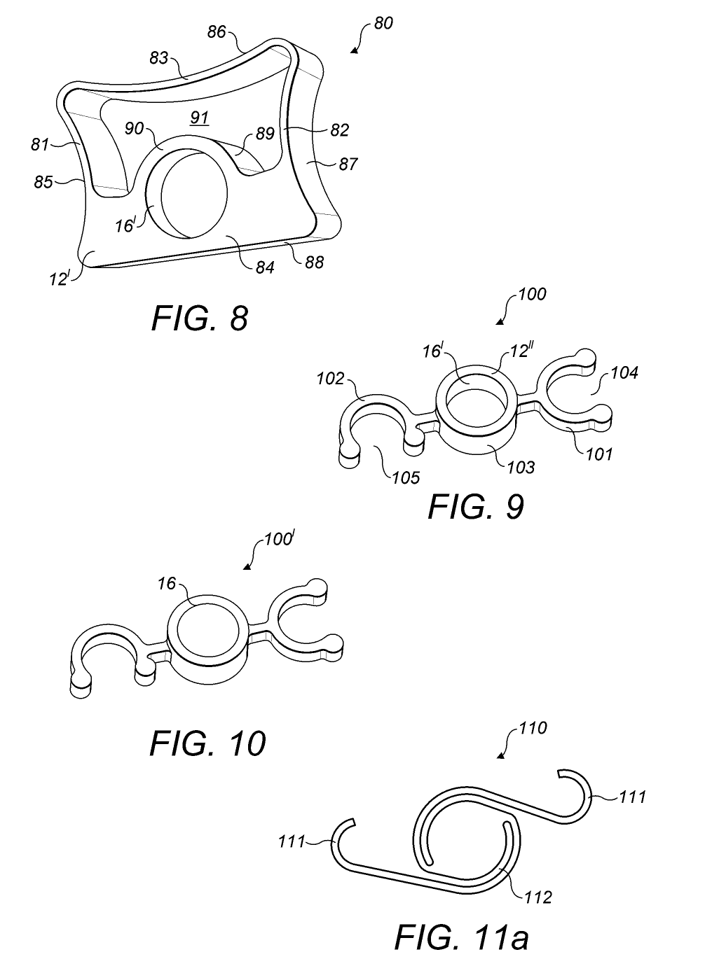

FIG. 8 shows a second embodiment of the clip, which comprises a recess for receiving a correspondingly formed projection;

FIG. 9 shows a further embodiment of the clip;

FIG. 10 shows a further embodiment of the clip, similar to that of FIG. 9;

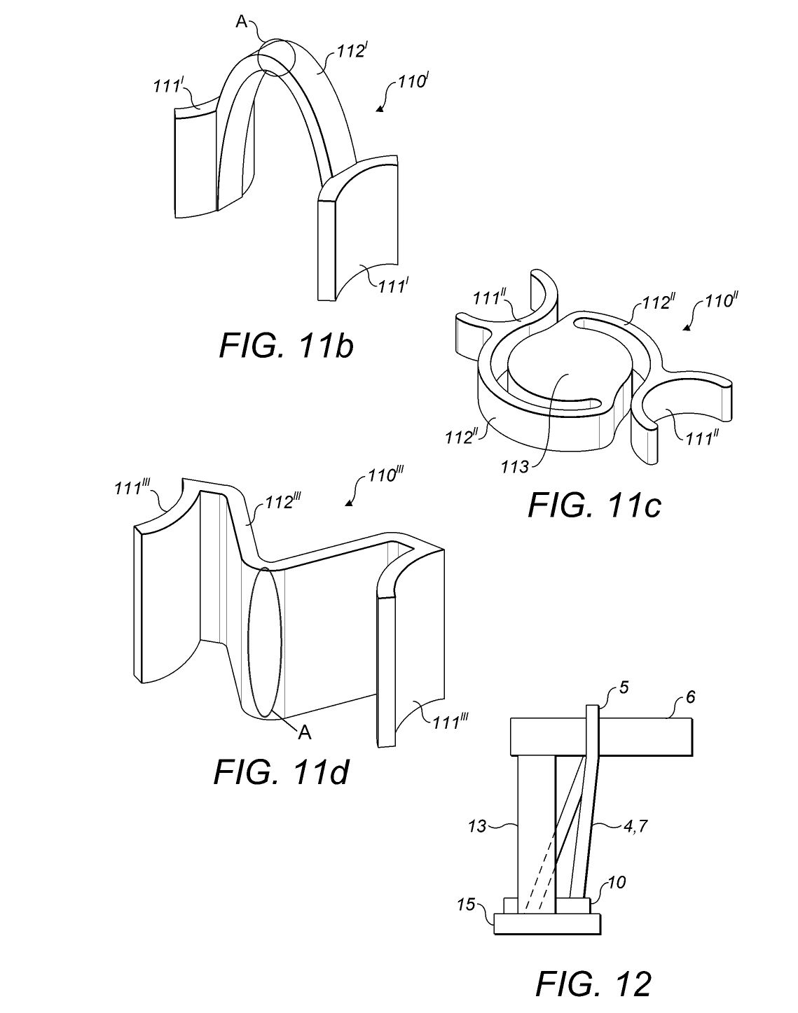

FIGS. 11a to 11d show further alternative embodiments of the clip; and

FIG. 12 shows the angle of the rod of an implantable device in relation to the plate, for two different configurations.

Components common to more than one figure or more than one embodiment are labelled in the figures using common reference numerals.

FIG. 1 illustrates a first embodiment of the present invention, in which the implantable device is an active middle ear implant or hearing actuator 1. The actuator 1 comprises an elongate transducer 2, which is formed by a stack of piezoelectric crystals 3. The transducer is housed in a frame 4, which is connected at one end to a super-elastic spring clip 5 for engaging the incus long process 6, and at the other end to a rod 7, which projects longitudinally from the end of the transducer and terminates in a rounded end or projection 9.

The actuator 1 further comprises a plate 10 which has a first substantially planar surface 11, and a second substantially planar surface 12 opposite said first surface. The plate 10 is configured to fit between the arches 13 of the stapes 22, with the first planar surface 11 substantially in contact with the exposed surface 14 of the stapes footplate 15.

A rounded recess or indentation 16 is formed in a central region of the second surface 12 of the plate 10, for engaging the projection 9. The rod 7 and the plate 10 are shown in cross section in FIG. 2. Both the projection and the recess have a substantially hemispherical form. The radius of curvature of the recess is fractionally larger than that of the projection, such that the projection is movable within the recess. Accordingly, when the projection is inserted in the recess, the end of the rod is held in position on the plate, whilst the angle of the rod relative to the plate is may be adjusted or varied.

To implant the actuator 1, a surgeon accesses the middle ear cavity 17 in a conventional manner. The plate 10 is located over the stapes footplate 15, in a central region between the stapes arches 13, with its first surface 11 in contact with the footplate, and held in place by the surgeon.

Specifically, the plate 10 is mounted on the stapes footplate 15 such that the recess 16 is located equidistant from each of the stapes arches 13, but offset from the central point between the arches.

The rounded end or projection 9 of the rod 7 is then located in the recess 16, to create a pivotal connection, about which the rod can be rotated until the spring clip 5 at the opposite end of the actuator meets the incus long process 6. The actuator 1 is then mounted to the incus long process by opening the jaws of spring clip 5 using tweezers, locating these around the incus long process, and then releasing the jaws.

In use, vibrations generated by the transducer are conducted through the rod 7 and the plate 10 to the stapes footplate 15, which in turn vibrates the oval window membrane 18 to generate pressure variations in the fluid filled cochlea 19.

Because the coupling between the rod 7 and the plate 10 is offset from the central point between the stapes, the actuator 1 extends substantially perpendicularly relative to the plate 10. This makes the transfer of vibrational energy from the actuator to the stapes footplate efficient, because the component of force parallel to the footplate is minimal.

This may be contrasted with the situation where the coupling between the rod 7 and the plate 10 is located over the central region between the stapes arches. In this case, the actuator 1 must be angled relative to the plate 10, to avoid the neck and head of the stapes. As a result, there is a significant component of force parallel to the footplate, making the transmission of vibrational energy less efficient.

The two configurations are compared in FIG. 12.

The position of the rod 7 with respect to the stapes footplate 15 is reliably maintained, through the engagement of the projection 9 in the recess 16, and through friction between the surface of the plate 10 and the exposed surface 14 of the footplate 15. Because the surface area of the plate in contact with the surface of the footplate is significantly larger than the contacting surface of the rod of the actuator disclosed in WO 2008/139225, the risk of the actuator slipping with respect to the footplate is significantly reduced. Accordingly, the actuator is effectively prevented from losing contact with the stapes footplate, or from slipping to a position on the stapes footplate in which vibrations generated by the actuator are conducted to the footplate less effectively.

The frame 4, spring clip 5, rod 7 and plate 10 are all formed of titanium or a nickel titanium alloy such as Nitinol, or other material with bioactive properties that encourage bone growth. Thus, over time, bone will grow to the plate, to secure it in position on the footplate 15, and eliminate any residual risk of the plate being dislodged.

The material from which the spring-clip 5 is formed, preferably a nickel titanium alloy such as Nitinol, is preferably treated to have super-elastic properties, such that the spring clip itself is super-elastic.

An alternative form of the rod 7′, suitable for engagement with the plate 10 of FIGS. 1 and 2 is shown in FIG. 3. In this embodiment, the rounded end or projection 9′ of the rod 7′ comprises a substantially hemispherical portion 30 with a diameter substantially the same as that of the rod. An annular groove 31 is cut into the rod, immediately adjacent the hemispherical portion.

The annular groove 31 increases the range of angular adjustment of the rod 7′ in relation to the plate 10, which facilitates implantation of the actuator 1 in the middle ear 17.

In particular, by forming an annular groove 31 immediately adjacent the hemispherical portion 30 of the rod 7′, the range of angular adjustment can be increased, without the requirement for the projection 9′ to have a diameter greater than that of the rod.

An alternative form of the recess 16′ suitable for engagement with the projection 9 or 9′ of FIGS. 1, 2 and 3 is shown in cross section in FIG. 4. In this embodiment, the recess is a cylindrical cavity in the plate 10′. In FIG. 4, the cylindrical opening extends only partially through the thickness of the plate. However, the cylindrical opening may extend through the full thickness of the plate, to form a circular opening on both the first and second surfaces 11, 12 of the plate.

In the embodiment of FIG. 1, the plate 10 is held in position on the stapes footplate through friction. However, in other embodiments, the plate may be held in place on the footplate 15 by means of a clip.

FIG. 5 illustrates a clip 50 suitable for attaching an implantable device such as the plate 10 of the implantable device 1 of FIG. 1 to the stapes footplate 15. For clarity, the clip is shown next to an illustration of the stapes 22.

The clip 50 comprises a continuous strip 51 of a super-elastic material, formed to have first and second curved sections 52, 53 for gripping the stapes arches 13, connected by a substantially M-shaped spring section 54.

The first and second curved sections 52, 53 are formed by respective ends of the strip 51, which are formed into substantially semi-circular or c-shaped arcs, aligned in a first plane, with their concave surfaces 55, 56 facing. At the end of each curved section, the strip extends away from the curved sections, in a second plane substantially perpendicular to the first plane, to form the spring section 54. Within this second plane, the strip extends from the respective curved sections, along a straight path 57 at an angle of approximately 80 degrees to the tangent of the semicircular arc at the end of the respective curved section. At a perpendicular distance from the curved sections which is approximately equal to the perpendicular distance from the footplate to neck of an average sized stapes, the strip is bent away from the respective curved sections through and angle of approximately 180 degrees, to form substantially semi-circular arcs 58. These semicircular arcs are connected by a substantially u-shaped section 59, the lowest part 60 of which substantially coincides with the intersection of the first and second planes.

The first and second curved sections 52, 53 are configured to correspond to the form of the outwardly facing surfaces 20 of the stapes arches 13. The connecting spring section 54 is formed to connect the curved sections such that, in the absence of external forces, the maximum distance between the concave surfaces 55, 56 of the curved sections is less than the distance between the outward facing surfaces of the stapes arches in a region where the these meet the footplate 15.

The connecting spring section 54 is also configured to allow the clip 50 to be resiliently deformed, to widen the gap between the curved sections 52, 53 by a sufficient distance to allow the curved sections to be passed around the stapes arches 13.

In particular, the M-shaped form of the connecting spring section, in which the M shape is relatively tall, ensures that the deflection in the regions indicated by circles A in FIG. 5 is sufficient for the spring section to operate in the super-elastic mode, where force is substantially constant over a wide range of deflection. The regions identified by the circles A are the regions which deflect the most when the clip is deformed to grip the stapes arches.

To mount the clip 50 on the stapes 22, the first and second curved sections 52, 53 of the clip are drawn apart by the surgeon, against the action of the connecting spring section 54, until the distance between the ends of the strip 51 becomes greater than the maximum distance between the outward facing surfaces 20 of the stapes arches 13. In this state, the first and second curved sections of the clip are passed around the respective arches, in a region where the arches meet the stapes footplate 15, with the clip oriented such that the first plane thereof is parallel to the surface 14 of the footplate. The ends of the clip are then released.

When the ends of the clip are released, the connecting spring portion draws the first and second curved sections together, to grip the respective arches of the stapes securely.

The super-elastic properties of the clip 50 mean that the curved sections 52, 53 exert a substantially constant force over a wide range of deflections of the connecting spring portion 54. Accordingly, a clip of given dimensions can accommodate significant variation in the dimensions of the stapes 22, without the risk of exerting too much, or too little force on the stapes arches 13.

When the clip 50 is mounted on the stapes, the first and second curved sections 52, 53 extend along, or close to the surface 14 of the footplate 15. The bottom 60 of the u-shaped section 59 of the connecting spring portion 54 also lies in contact with, or close to the footplate 15. The u-shaped section thus provides a surface to which another component can be mounted, to contact the stapes footplate.

For example, the clip 50 of FIG. 5 may be used to couple a plate, such as the plate 10, 10′ of FIGS. 1, 2 and 3, to the stapes footplate 15. In this case, the plate may be bonded to a bottom section 60 of the u-shaped section 59 of the clip, as shown in FIG. 6. Thus, when the clip is mounted to the stapes arches 13, in the manner described above, the plate will be located over and in contact with the stapes footplate.

FIGS. 7a to 7c respectively show front, rear and perspective views of an embodiment of the present invention which takes the form of a middle ear implant 1′. The implant 1′ comprises a rod and plate arrangement as described above in relation to FIGS. 1 to 3, wherein the plate 10 is mountable to the stapes footplate by means of the clip 50 illustrated in FIGS. 5 and 6.

The implant comprises a super-elastic spring clip 5′ for engaging the incus long process. The spring clip is connected by a rod to a first side of a housing 4′ for a transducer element (not shown). A rod 7′ extends from a second side of the housing, opposite said first side, and terminates in a substantially hemi-spherical projection 9′. An annular groove 31 is formed in the rod immediately adjacent hemispherical portion 30 of the projection.

The projection 9′ is located in a correspondingly formed cavity 16 formed in a central region of a plate, such that the rod 7′, and thus the housing 4′ and the spring clip 5′ are pivotally mounted on the plate 10.

A side face 70 of the plate 10, perpendicular to one of the first and second surfaces 11, 12 thereof, is welded or otherwise bonded to the clip 50. Specifically, the side face of the plate is bonded to the lowest region 60 of the u-shaped section 59 of the clip.

To implant the device 1′ illustrated in FIGS. 7a-7c, the curved sections 52, 53 of the clip 50 are located around the stapes arches 13, as described above in relation to FIGS. 5 and 6, such that the plate 10 lies over and in contact with the stapes footplate 15 in a central region thereof.

The projection 9′ at the end of the rod 7′ is then located in the recess 16, and the implant 1′ is rotated into position, as described above in relation to the embodiment of FIG. 1.

In other embodiments of the present invention, the recess which receives the projection at the end of the rod may be formed in an integral part of the clip.

FIG. 8 shows a clip 80 for attachment to the stapes arches, which comprises a cylindrical recess 16′ for receiving a correspondingly formed projection.

The clip 80 comprises two relatively shorter side sections 81, 82 and two relatively longer side sections 83, 84, which together form a substantially rectangular frame.

The two shorter side sections 81, 82 and one of the longer side sections 83 are relatively thin, and curve inwards to form three concave outer edges 85, 86, 87 of the frame. The fourth side section 84 is relatively wider, and has a straight edge which forms the fourth outer edge 88 of the frame, and a curved inner edge 89 which defines a substantially semicircular portion 90 which projects into the opening 91 defined by the frame edges 85-88.

The fourth side section 84 thus forms a plate-like region of the clip, and has first and second planar surfaces 11′, 12′, perpendicular to the frame edges 85-88. A cylindrical recess 16′ is formed in the second surface 12′ of the plate-like region, substantially centrally between the two shorter side sections 81, 82, such that the recess is partially located in the semicircular portion 90, closer to the straight outer edge 88 and the curved inner edge 89. The cylindrical recess extends through substantially half the thickness of the clip.

The two shorter side sections 81, 82 of the clip 80 are configured to correspond to the form of the inward facing surfaces 21 of the stapes arches 13, and are spaced apart such that the shortest distance between their concave surfaces is slightly longer than the distance between the inward facing surfaces of the stapes arches, in a region immediately above the footplate.

The clip 80 is formed of a super-elastic material, and can thus be resiliently deformed to pass through the stapes arches 13, and exert a substantially constant force on the stapes for different stapes sizes.

To mount the clip 80 to the stapes 22, the surgeon deforms the clip by moving the longer side edges 83, 84 towards one another in a central region of the clip. This action draws the shorter side sections 81, 82 together at the side of the frame where they meet the first longer side section 83, so that this side of the clip can be inserted between the stapes arches 13.

In this state, the clip 80 is inserted through the stapes arches 13, and positioned such that the first surface 11′ of the plate region rests on the surface 14 of the footplate 15.

The clip 80 is then released by the surgeon, such that the shorter side edges 81, 82 spring apart to grip the inward facing surfaces 21 of the stapes arches 13, and thereby hold the plate-region in position on the footplate 15.

As with the embodiment of FIGS. 5 and 6, the super-elastic properties of the clip mean that the curved sections exert a substantially constant force over a wide range of deflections of the connecting spring portion. Accordingly, a clip of given dimensions can accommodate significant variation in the dimensions of the stapes arch, without the risk of exerting too much, or too little force on the stapes arches.

FIG. 9 shows another stapes clip 100 for attachment to the stapes arches 13, which comprises a cylindrical recess 16′ for receiving a correspondingly formed projection.

The stapes clip 100 comprises first and second c-shaped sections 101, 102 connected either side of a central connecting region 103 for engaging the stapes arches 13.

The central connecting region 103 is substantially circular, and has a first planar surface 11>> for contacting the stapes footplate 15, and a second planar surface 12>> opposite said first surface, in which a cylindrical recess 16> is formed.

The right hand c-shaped section 101, as viewed in FIG. 9, defines a major arc of a circle, the ends of which are spaced apart by a distance which is larger than the diameter of a single stapes arch 13, to form an opening 104, which is perpendicular to the longitudinal axis of the clip 100. The right hand c-shaped section can thus slide over a first one of the stapes arches.

The left hand c-shaped section 102, as viewed in FIG. 9, also defines a major arc of a circle. The ends of this arc form an opening 105 which is parallel to said longitudinal axis. The left hand c-shaped section is wide enough to slide over a second one of the stapes arches, when the right hand c-shaped section is located around a first one of the arches. The left hand c-shaped section may also be made wide enough to accommodate a range of distances between the stapes arches.

To mount the stapes clip 100 to the stapes 22, the right hand c-shaped section 101 slides over a first one of the stapes arches 13 in a region where the arch meets the footplate 15. Having mounted the right hand spring c-shaped section 101 on the first stapes arch, the surgeon rotates the clip 100 anti-clockwise until the left hand c-shaped section 102 extends around the second stapes arch to hold the clip in place. In this configuration, the clip 100 can no longer rotate relative to the stapes 22 without disengaging the left hand c-shaped section 102, and the first surface 11″ of the central connecting portion 103 lies in contact with a central region of the stapes footplate 15.

A suitably formed projection can then be located in the cylindrical recess 16′ to couple a further element to the stapes footplate 15, in the manner described above in relation to FIGS. 1 to 4.

FIG. 10 shows a similar clip 100′ to that of FIG. 9, in which the cylindrical recess 16′ is replaced by a substantially hemi-spherical recess 16.

Alternative stapes clips 110, 110′, 110″, 110′″ for coupling an implantable device to the stapes footplate are illustrated in FIGS. 11a to 11d. Each of these embodiments comprise curved sections 111, 111′, 111″, 111′″ for engaging the stapes arches in the region where they meet the footplate, and a connecting spring section 112, 112′, 112″, 112′″, which allows the clip to be resiliently deformed to pass around or between the stapes arches, and to press against the stapes arches to hold the spring in place.

In particular, in the embodiment of FIG. 11a, the clip comprises a coiled spring 112 the ends of which can be pulled outwards to wrap the curved sections 111 around the stapes arches. When the clip is located between the stapes arches, the coiled spring tries to revert to its original configuration, causing the curved sections to press against the outwardly facing surfaces of the stapes arches, to hold the clip in place on the stapes footplate.

In the embodiment of FIG. 11c, the connecting spring section 112″ comprises first and second curved beams, respectively connected to first and second curved sections. The curved sections 111″ can be pushed inwards deforming the curved beams 112″, to allow the clip to be inserted between the stapes arches. When the clip is located between the stapes arches, the curved beams try to revert to their original positions, causing the curved sections 111″ to press against the inward facing surfaces of the stapes arches, to hold the clip in place on the stapes footplate.

A plate or other element can be bonded or otherwise mounted to each of the clips of FIGS. 11a to 11d, for coupling the clip to a further implantable element. Moreover, a rounded recess may be formed in central section 113 of the clip of FIG. 11c for receiving a correspondingly formed projection.

In general, the resilient clips or attachment means of the present invention are configured to deflect sufficiently when deformed from their original or natural configuration to a configuration in which the engagement sections engage the stapes arches, such that the clip engages the stapes arches super-elastically. As a result, the force exerted on the arches by the engagement sections of the clip is substantially constant over a wide range of deflection. This ensures that a clip of a given size can accommodate significant anatomical variation between patients.

This may be achieved by configuring the attachment means to ensure sufficient deflection of specific regions of the connecting spring section of the clip, when the clip is deformed to engage the stapes arches.

For example, the clip 50 of FIG. 5 is configured such that the regions identified by circles A deflect sufficiently when the clip is deformed to engage the stapes arches, for the clip to operate in the super-elastic range. As a further example, the clip 111′ of FIG. 11b is configured such that the region identified by circle A deflects sufficiently when the clip is deformed to engage the stapes arches, for the clip to operate in the super-elastic range. As yet another example, the clip 111′″ if FIG. 11d is configured such that the region identified by oval A deflects sufficiently when the clip is deformed to engage the stapes arches, for the clip to operate in the super-elastic range.

The present invention has been described above in terms of a hearing actuator which extends from the incus long process to the stapes footplate. However, the principles of the present invention apply equally to other types of implant, both active and passive, which are configured to extend to the stapes footplate from other parts of the middle ear, or locations outside the middle ear. Preferably, however, the implant of the invention is configured to extend to the stapes footplate from a second attachment region on an element other than the stapes itself.

The present invention has also been described in terms of embodiments wherein an opening or recess is formed on a footplate engaging portion, and a corresponding projection is formed at the end of an elongate portion of the implant. However, it will be appreciated that the recess could be formed on the elongate portion of the implant, and the projection could be formed on the footplate engaging portion.

1. An implantable device for implantation in the middle ear, the device comprising engagement means for engaging the footplate of the stapes, the engagement means comprising:—
a first coupling portion configured for location on the footplate of the stapes; and
a second coupling portion for coupling to the first coupling portion;
wherein one of the first and second coupling portions comprises a projection, and the other of said portions comprises a correspondingly formed opening for receiving the projection, to provide a pivotal coupling between said first and second coupling portions.

2. An implantable device according to claim 1 wherein the projection is formed on the second coupling portion and the corresponding opening is formed on the first coupling portion.

3. An implantable device according to claim 1 or 2 wherein the projection is formed on the first coupling portion and the corresponding opening is formed on the second coupling portion.

4. An implantable device according to any preceding claim wherein the projection is a rounded projection.

5. An implantable device according to any preceding claim wherein the projection has a substantially constant radius of curvature.

6. An implantable device according to any preceding claim wherein the projection has a substantially hemispherical form.

7. An implantable device according to any preceding claim wherein the portion on which the projection is formed comprises an annular groove adjacent the projection.

8. An implantable device according to any preceding claim wherein the opening has a substantially constant radius of curvature.

9. An implantable device according to any preceding claim wherein the opening has a substantially hemispherical form.

10. An implantable device according to any preceding claim wherein the radius of curvature of the projection is fractionally smaller than the radius of curvature of the opening.

11. An implantable device according to any of claims 1 to 7 wherein the opening has a substantially cylindrical form.

12. An implantable device according to any preceding claim wherein the implantable device comprises attachment means for attaching the first coupling portion to the stapes footplate.

13. An implantable device according to claim 12 wherein the attachment means comprises first and second engagement sections connected by a connecting portion, wherein each of the engagement sections has an engagement surface configured to engage a respective one of the stapes arches.

14. An implantable device according to claim 13 wherein the engagement surface of the first engagement section faces in an opposite direction to the engagement surface of the second engagement section.

15. An implantable device according to claim 14 wherein the attachment means is configured such that the engagement surfaces face towards one another.

16. An implantable device according to claim 14 wherein the attachment means is configured such that the engagement surfaces face away from one another.

17. An implantable device according to any of claims 13 to 16 wherein the engagement surfaces are spaced apart by a distance substantially equal to the distance between the stapes arches where they meet the stapes footplate.

18. An implantable device according to any of claims 13 to 17 wherein the engagement sections each comprise a curved section.

19. An implantable device according to any of claims 13 to 18 wherein said engagement surfaces are concave surfaces.

20. An implantable device according to any of claims 13 to 19 wherein the attachment means is at least partially resilient, to be deformable between a first configuration for insertion through the stapes arches or passing around the stapes arches, and a second configuration for engaging the stapes arches.

21. An implantable device according to any of claims 13 to 20 wherein the attachment means has super-elastic properties.

22. An implantable device according to any of claims 13 to 21 wherein the connecting portion comprises two resilient sections, located one on each side of a central region of the connecting portion.

23. An implantable device according to any of claims 13 to 22 wherein the first coupling portion is integrally formed with the connecting portion of the attachment means.

24. An implantable device according to any of claims 13 to 23 wherein the first and second engagement sections extend in a first plane, and the connecting portion extends from the engagement sections in a second plane perpendicular to the first plane.

25. An implantable device according to any of claims 13 to 24 wherein the device is configured such that the opening or projection of the first coupling portion is located substantially equidistant from the engagement surfaces of the attachment means.

26. An implantable device according to claim 25 wherein the opening or projection is offset from a central point between the engagement surfaces.

27. An implantable device according to any preceding claim wherein the device is formed from a biocompatible material.

28. Attachment means for attaching an implantable device to the stapes footplate, the attachment means comprising first and second engagement sections connected by a connecting portion, wherein each of the engagement sections has an engagement surface configured to engage a respective one of the stapes arches.

29. Attachment means according to claim 28 wherein the engagement surface of the first engagement section faces in an opposite direction to the engagement surface of the second engagement section.

30. Attachment means according to claim 28 or 29 wherein the engagement sections are configured such that the engagement surfaces face towards one another.

31. Attachment means according to claim 28 or 29 wherein the engagement sections are configured such that the engagement surfaces face way from one another.

32. Attachment means according to any of claims 28 to 31 wherein the engagement surfaces are spaced apart by a distance substantially equal to the distance between the stapes arches where they meet the stapes footplate.

33. Attachment means according to any of claims 28 to 32 wherein the engagement sections each comprise a curved section.

34. Attachment means according to any of claims 28 to 33 wherein said engagement surfaces are concave surfaces.

35. Attachment means according to any of claims 28 to 34 wherein the attachment means is at least partially resilient, to be deformable between a first configuration for insertion through the stapes arches or passing around the stapes arches, and a second configuration for engaging the stapes arches.

36. Attachment means according to any of claims 28 to 35 wherein the attachment means has super-elastic properties.

37. Attachment means according to any of claims 28 to 36 wherein the connecting portion comprises two resilient sections, located one on each side of a central region of the connecting portion.

38. Attachment means according to any of claims 28 to 37 wherein the first and second engagement sections extend in a first plane, and the connecting portion extends from the engagement sections in a second plane perpendicular to the first plane.

39. Attachment means according to any of claims 28 to 38 further comprising a footplate engaging portion for location on the footplate of the stapes.

40. Attachment means according to claim 39 wherein the footplate engaging portion is integrally formed with the attachment means.

41. Attachment means according to claim 40 wherein the footplate engaging portion comprises one of an opening and a projection for receiving a correspondingly formed opening or projection of a coupling portion, to form a pivotal coupling with said coupling portion.

42. Attachment means according to claim 41 wherein the attachment means is configured such that said opening or projection is located substantially equidistant from the first and second engagement surfaces.

43. Attachment means according to claim 42 wherein the opening or projection is offset from the central point between the engagement surfaces.

44. Attachment means according to any of claims 41 to 43 wherein the attachment means further comprises a coupling portion for coupling with the footplate engaging portion, wherein the coupling portion comprises said correspondingly formed opening or projection for forming the pivotal coupling with the footplate engaging portion.

45. Attachment means according to claim 44 wherein the projection is formed on the footplate engaging portion and the opening is formed on the coupling portion.

46. Attachment means according to claim 44 wherein the projection is formed on the coupling portion and the opening is formed on the footplate engaging portion 4.

47. Attachment means according to any of claims 28 to 46 wherein the attachment means is formed from a bio-compatible material.

48. A method of mounting an implantable device to the stapes footplate, the method comprising:—
locating a first coupling portion on the stapes footplate;
locating a second coupling portion on the first coupling portion, to be pivotally coupled therewith through the engagement of a projection formed on one of said first and second coupling portions and a correspondingly formed opening formed in the other of said portions; and
adjusting the angle of the second coupling portion to a desired position.

49. A method of attaching an implantable device to the stapes footplate, the method comprising:—
providing an attachment means having first and second engagement sections, each configured to engage a respective one of the stapes arches; and
locating the first and second engagement sections around the respective stapes arch adjacent the stapes footplate.

50. An implantable device for implantation in the middle ear, substantially as hereinbefore described with reference to the accompanying drawings.

51. An attachment means substantially as hereinbefore described with reference to the accompanying drawings.

52. A method of mounting an implantable device to the stapes footplate, substantially as hereinbefore described with reference to the accompanying drawings.

53. A method of attaching an implantable device to the stapes footplate, substantially as hereinbefore described with reference to the accompanying drawings.
